(19) United States
(12) Patent Application Publication
(10) Pub. No.: US 2003/0108532 A1
(43) Pub. Date: Jun. 12, 2003

(54) Methods for identifying peptide aptamers capable of altering a cell phenotype

(75) Inventors: John D. Benson, West Roxbury, MA (US); Sylvie Maglie Vincent, Somerville, MA (US); Bradley Bryan Brasher, Natick, MA (US)

Correspondence Address: LAHIVE & COCKFIELD, 28 STATE STREET, BOSTON, MA, 02109, US

(73) Assignee: Enanta Pharmaceuticals, Watertown, MA

(21) Appl. No.: 10/263,577

(22) Filed: Oct. 3, 2002

Related U.S. Application Data
(63) Continuation of application No. PCT/US01/10953, filed on Apr. 4, 2001.
(60) Provisional application No. 60/194,722, filed on Apr. 4, 2000.

(51) Int. Cl. G01N 33/574; C12Q 1/68; A61K 48/00; C12N 15/867

(52) U.S. Cl. 424/93.21; 435/6; 435/7.23; 514/44; 435/456

(57) ABSTRACT

The invention provides methods and compositions for screening and identifying peptide aptamers that can modulate a cell phenotype and further, can be used for the treatment of a disease involving a misregulated cell phenotype, such as, for example, a cancer.

### RELATED INFORMATION

The present application is a continuation of PCT patent application number PCT/US01/10953, filed on Apr. 4, 2001, which claims priority to U.S. provisional patent application No. 60/194,722, filed on Apr. 4, 2000, the entire contents both of which are hereby incorporated by reference.

### BACKGROUND OF THE INVENTION

Cancer is the second leading cause of death in the United States. Nationally, 1.2 million new cases of cancer are diagnosed each year, accounting for approximately 550,000 deaths. In 1997, cancer care, including screening, diagnosis, treatment, and supportive care, was estimated to consume approximately 15% of all health care costs in the United States (Gerszten, 1997). For the fiscal year 2000, the National Cancer Institute (NCI) estimates the overall costs for cancer at $107 billion; $37 billion for direct medical costs, $11 billion for indirect morbidity costs (lost productivity due to illness), and $59 billion for indirect mortality costs. The combined annual research budgets for the NCI and the American Cancer Society (ACS) for the fiscal year 2001 will exceed $3 billion.

Tangible progress in cancer therapy has lagged in proportion to the resources and efforts that have been expended. According to NCI and ACS statistics, the overall five-year survival rate for all cancers is estimated to be 59%. Contrary to popular conception, this is an overall improvement of less than 10% since 1974. Upon recent celebration of the fiftieth anniversary of cancer chemotherapy (Curt, 1996), the hundred or so widely used cancer therapeutic drugs in clinical use had been known and used for many years. However, the mechanism of action of these mainstay drugs has only recently begun to be appreciated. It was long believed that these mainstay chemotherapeutic agents directly affected metabolic activities associated with cellular DNA replication or cell division. It is now known that when these compounds are effective; they most often induce apoptosis, and do so by acting on targets that are only now being identified.

Based on the long-noted toxicity and lack of classical chemotherapeutic agents (e.g. cisplatin, 5-fluorouracil, hydroxyurea, vincristine and their derivatives), it has been hoped that an understanding of the molecular and genetic differences between normal and cancerous cells would suggest ways of killing cancer cells with greater selectivity and efficiency. Analysis of the molecular basis of cancer and the regulation of cell division, an ongoing energetic pursuit for almost thirty years, has identified hundreds of potential targets-genes and their protein products that form the conceptual basis for cancer therapeutic drug discovery or design. These include oncogenes, tumor suppressor genes, as well as the regulators and components of the mitotic, cell division, or apoptotic activities of the cell. Indeed, since 1982, almost 200 genes have been identified that are classified as tumor suppressors or oncogenes in the Online Mammalian Inheritance in Man (OMIM) database of the National Center for Biotechnology Information (NCBI). Of these genes, and the proteins they encode, only two of these targets have thus far yielded therapeutically proven small molecule drugs: small molecule inhibitors of farnesyl transferase, which inhibit function of the ras oncogene by preventing its post-translational modification and localization of ras to the inner cell membrane (Zujewski et al., 2000), and an inhibitor of the abl kinase associated with the bcr-abl Philadelphia chromosomal translocation (Carroll et al., 1997).

The difficulty in understanding cancer is no doubt due to the fact that the regulatory systems governing cell proliferation and apoptosis are extremely complex. Cellular alterations that lead to cancer are highly diverse, and may require multiple epigenetic changes even within a single cell (reviewed in Boland and Ricciardiello, 1999; Hanrahan and Weinberg, 2000).

One approach to the investigation of complex biological systems is to use combinatorial chemistry to synthesize diverse compound libraries that are screened for phenotypic effects in cells. Just as screens for the phenotypic effects of mutations served as an initial step in the characterization of basic metabolic and regulatory pathways in lower organisms several decades ago (i.e. in fungi and bacteria), it is believed that this approach may provide powerful means of examining the highly complex regulatory networks and pathways in mammalian cells. There are two crucial components to such an approach: (i) establishment of screening assays that allow phenotypic analysis of several million compounds, and (ii) development of highly diverse compound libraries in a format that allows molecular identification of the effective compound (deconvolution).

Both of these requirements are inadequately met by current technologies. The largest deconvolutable combinatorial chemical libraries that presently exist in tenable screening formats constitute one to two million compounds (Tan et al., 1998). Moreover, although phage display libraries represent a greater source of combinatorial diversity (i.e. 10⁹ different molecules in libraries composed of seven random natural amino acids), screening of these libraries is limited to evaluation of binding to known and specified target molecules. Screening only for binding does not immediately consider whether ligand binding affects a function of the target. In addition, since foreknowledge of a particular pathway and its components is required for the design of such binding screens, this approach is applicable only to targets within relatively well understood pathways.

Accordingly, the need still exists for improved methods to identify compounds capable of modifying cellular pathways for treating diseases, such as cancer.

### SUMMARY OF THE INVENTION

The present invention provides an efficient high-throughput system for the molecular analysis of cells, leading to the identification of novel peptides (aptamers) that function intracellularly, and that manifest identifiable phenotypes in a eukaryotic cell, e.g., mammalian systems. Optimized vectors, e.g., retroviral vectors, are used to transduce libraries expressing random peptides in a variety of cell types, preferably mammalian (e.g., human). These libraries are then used to screen tens of millions of aptamers in one experiment using any appropriate cell type, followed by identification of validated active aptamers by sequencing at the end of the screen. Thus, the present invention surpasses existing research strategies that rely on target identification and selection, including those based on elucidation of specific protein-protein interactions, phenotypic gene expression profiling, or genotypic analysis. This is especially advantageous in the study of a complex and highly diverse disease such as cancer.

Accordingly, the present invention provides several advantages over current methods for identifying therapeutic peptides that include, but are not limited to, the following:

providing an assay that can identify from a highly diverse library an aptamer capable of modulating a specified cell phenotype;

providing an assay that can probe an extremely complex cellular phenotype (e.g., cancer) without any a priori knowledge regarding the underlying mechanism of the cellular event;

providing an assay system that has a strong output signal that can be distinguished from a background of little or no signal;

providing an assay system that can measure a cell phenotype as a quantifiable signal or a qualitative signal involving variable cell growth or other biological effect;

providing an assay that contains a control which accurately identifies peptide aptamers that affect identifiable cell lines but not normal cells to insure that inappropriate aptamers are not further investigated and that candidate aptamers are not erroneously dismissed. In the case of cancer cells, aptamers that confer an apoptotic phenotype in cancerous, but not in non-cancerous cells is desired.

Accordingly, in one aspect, the present invention provides a method for identifying a peptide aptamer capable of modifying a cell phenotype by a) contacting cells with a library of expressible nucleic acid sequences encoding random peptide aptamers; b) selecting at least one cell having an altered phenotype compared to the phenotype of the cell prior to the contacting step (a); and c) identifying at least one or more peptide aptamers expressed in the selected cell.

The method can further include amplifying the nucleic acid sequences identified in step c) and repeating one or more times steps a)-c) using the amplified nucleic acid sequences as the library of expressible nucleic acid sequences. Preferably the nucleic acid sequences are amplified using the polymerase chain reaction (PCR) and a thermostable nucleic acid polymerase.

In a particular embodiment, the method is used to identify a peptide aptamer capable of altering a cell phenotype associated with a change in levels of apoptosis, signal transduction, protein trafficking, cell adhesion, membrane transport, cell motility, or differentiation.

In a related embodiment, the peptide aptamer is identified by selecting a cell having an altered phenotype by measuring a change in levels of apoptosis, signal transduction, protein trafficking, cell adhesion, membrane transport, cell motility, or differentiation, the change in levels of apoptosis being measured using, e.g., immunohistochemistry. In another related embodiment, the peptide aptamer is identified by selecting a cell having an altered phenotype by measuring a change in levels of signal transduction, preferably mediated by a tyrosine kinase or a G protein coupled receptor or effectors thereof.

Suitable cells for use in the methods of the invention include, for example, fungal cells (e.g., yeast cells), insect cells, and mammalian cells, preferably human cells, and more preferably, clonal human cancer cells, or cells modified to exogenously express receptors or effectors of signal transduction. To transduce the cells, a library of expressible nucleic acid sequences encoding random peptide aptamers that are encoded in a eukaryotic expression vector, preferably, a retroviral vector, can be used.

Peptide aptamers of the invention generally comprise between 5-9 (e.g., 5, 6, 7, 8, or 9) amino acid residues or more. The peptide aptamer also can be fused to an additional amino acid sequence, such as thioredoxin, a regulatory polypeptide involved in apoptosis, bcl-2, p53, an NFκB-related polypeptide, a caspase, PTEN, myc, a BH3 domain, a death domain (DD), a BIR3 domain, a BIR domain, a nuclear localization signal sequence, a membrane localization signal sequence, a farnesylation signal sequence, a transcriptional activation domain, a transcriptional repression domain, and functional fragments thereof.

The present invention also provides a peptide aptamer, derivative thereof, or corresponding nucleic acid, identified according to the previously described methods, as well as pharmaceutical compositions containing the peptide aptamer, derivative thereof, or corresponding nucleic acid, in an acceptable carrier or diluent. These peptide aptamers, derivatives thereof, and corresponding nucleic acids, can be used according to art recognized techniques for the molecular modeling of an agent having similar structural and/or functional characteristics as the identified peptide aptamer.

Alternatively, the peptide aptamer, derivative thereof, or corresponding nucleic acid, can be used to treat a disease or condition associated with an aberrant (e.g., misregulated) cell phenotype in a subject by administering to the subject, a therapeutically effective amount of a peptide aptamer, derivative thereof, or corresponding expressible nucleic acid (e.g., by gene therapy) identified according to the previously described methods. In a related embodiment, the misregulated cell phenotype is associated with altered apoptosis, signal transduction, protein trafficking, cell adhesion, membrane transport, cell motility, differentiation, or a disease or condition such as cancer, osteoporosis, or hematochromatosis. In another related embodiment, the method involves administering the peptide aptamer using a retrovirus containing a nucleic acid sequence encoding the peptide aptamer.

In yet another embodiment, the invention provides a viral vector encoding a peptide aptamer identified according to the methods described above.

In still another embodiment, the invention provides a kit for screening a library of expressible nucleic acid sequences encoding peptide aptamers, or a panel of peptide aptamers, and instructions for use.

Other features and advantages of the invention will be apparent from the following detailed description and claims.

### BRIEF DESCRIPTION OF THE DRAWINGS

FIG. 1 is a schematic representation of how retroviral aptamer libraries can be generated. Typically, random oligonucleotides encoding peptide aptamer coding sequences of, e.g., seven amino acids, are incorporated into a plasmid vector that produces infectious retrovirus upon transfection into appropriate packaging cell lines (see text for more detail) in order to generate a library with a complexity of, e.g., greater than 10⁷. The aptamer may be fused to a translational regulatory leader and/or a fusion moiety if desired. Aptamer expression is driven by a strong promoter, e.g., the CMV or SV40 promoter; Ψ sequences within the plasmid vector direct retroviral packaging; and, although not shown, these plasmid vectors are also designed to encode resistance markers, such as, e.g., G418 and/or puromycin in order to facilitate culturing, selection, and retroviral titering.

FIG. 2 is a schematic representation of how peptide aptamers that are capable of preferentially inducing apoptosis in cancerous cells, but not normal cells, can be screened and identified. Using an exemplary cell line such as the myeloid leukemia cell line HL-60, the library is introduced under conditions that permit aptamer expression and selection of apoptotic cells. Aptamers identified as causing this phenotype in the cancerous HL-60 cells (represented as aptamers G and J) are then amplified by PCR and tested in normal cells (e.g., primary human fibroblasts) to insure that the aptamers will not harm normal cells.

FIG. 3 is a schematic representation of how other peptide aptamers can be identified as causing a desired phenotype (e.g., apoptosis) in other cancerous cell type (i.e., HeLa cells) as shown in FIG. 2. In addition, the schematic shows how aptamers identified as causing a particular phenotype in one cancerous cell line can be re-screened in other cancer cell lines. In particular, the schematic illustrates how aptamers can be screened for apoptotic activity in a large panel of cancer cell lines in order to determine the range and cell type specificity of the identified aptamer. For example, a retrovirus expressing a given previously identified aptamer, e.g., aptamer “J” determined to induce apoptosis in HL-60 cells, can be used to infect a diverse panel of other cancer cell types to assess its range of action.

FIG. 4 shows an exemplary panel of cell lines and identified aptamers having different effects that can be assembled into a data bank using the methods of the invention. In particular, the approach shown in FIG. 3 is repeated iteratively, until a set of aptamer-encoding retroviruses that are identified as having activity when tested against a broad variety of cancer cell lines in the cell panel is obtained. This allows for the selection of an aptamer that can be “tailored” for the treatment of a particular cancer cell type. The aptamer panel, shown as circles labeled “A-L”, can also be expressed in tumor biopsy cells, in order to phenotype these samples and generate a descriptive profile of these cells.

FIG. 5 is a schematic representation of a method for using aptamers to “functionally phenotype” cancer cells. For example, the method of the invention uses apoptotic aptamers identified in the iterative process shown in FIG. 2 to generate a profile of altered cellular phenotypes using a panel of different functional assays that can determine, e.g., surface molecule expression (FACS sorting), cell panning (adhesion), cell motility, and gene activation. The particular pattern of the cells susceptibility when expressing a certain aptamer is the “functional phenotype” of a given cell.

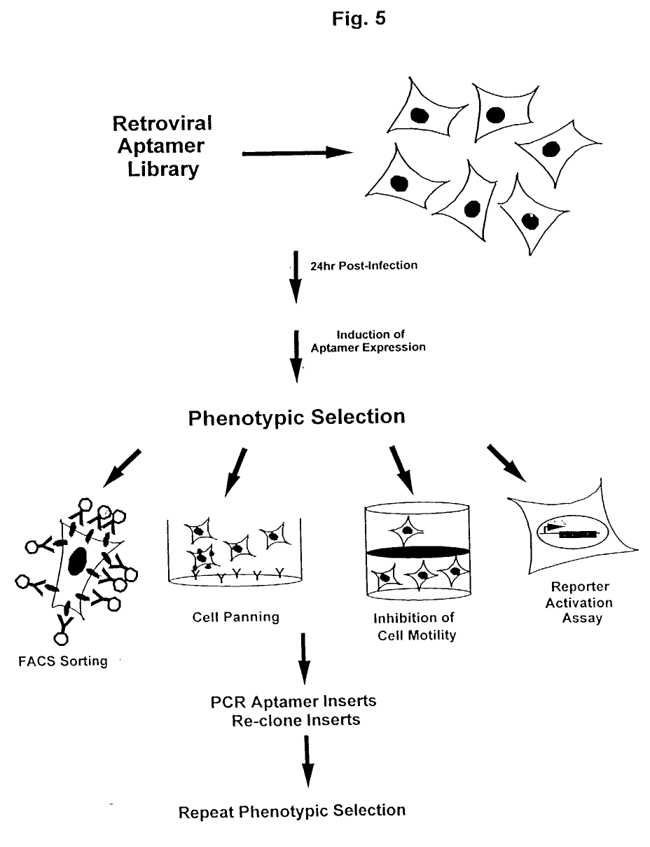

### DETAILED DESCRIPTION OF THE INVENTION

In order to provide a clear and consistent understanding of the specification and claims, including the scope to be given such terms, the following definitions are provided.

Definitions

As used herein the term “aptamer” or “peptide aptamer” refers to a polypeptide, generally between 2-20, preferably between 5-10 (i.e., 5, 6, 7, 8, 9, or 10), most preferably between 6-8 (e.g., 7) amino acid residues in length, capable of modifying the phenotype of a cell when introduced into or expressed in the cell.

The term “library of expressible nucleic acid sequences encoding random peptide aptamers” refers to a collection or plurality of nucleic acid sequences that encode different peptide aptamers (either alone or fused to other polypeptide sequences). Peptide aptamers differ randomly by one or more amino acids. The term “random” means that the differing sequences are not predetermined. Typically, the nucleic acid sequence/s are contained within a vector, for example, a plasmid, that can be propagated in a host cell, e.g., a prokaryotic host and can also be used to transfect or infect a eukaryotic cell. The terms “vector”, “vector construct”, “expression vector”, and “plasmid” are used interchangeably. The term “vector” also includes viral vectors, such as retroviral vectors derived from retroviruses.

The term “retroviral vector” refers to a vector suitable for propagating and/or expressing a nucleic acid sequence (e.g., a peptide aptamer sequence) in a cell (e.g., eukaryotic cell) which is derived, in whole or in part, from a retrovirus. Retroviral vectors are, in turn, generated by transfecting a pre-constructed plasmid library into an appropriate retroviral packaging cell lines.

The term “effector” refers to a naturally-occurring cell-associated (e.g., endogenous) polypeptide that is, directly or indirectly, responsible for a cellular phenotype.

The term “cell phenotype” includes any detectable aspect of a cell, such as the visual appearance or molecular function of the cell.

The term “cancer” includes any neoplasm, such as a carcinoma (derived from epithelial cells) or sarcoma (derived from connective tissue cells) or a cancer of the blood, such as a leukemia.

The term “apoptosis” refers to any non-necrotic, cell-regulated form of cell death, as defined by criteria well established in the art.

The terms “aberrant” and “misregulated” refer to a cell phenotype which differs from the normal phenotype of the cell, particularly those associated either directly or indirectly with disease.

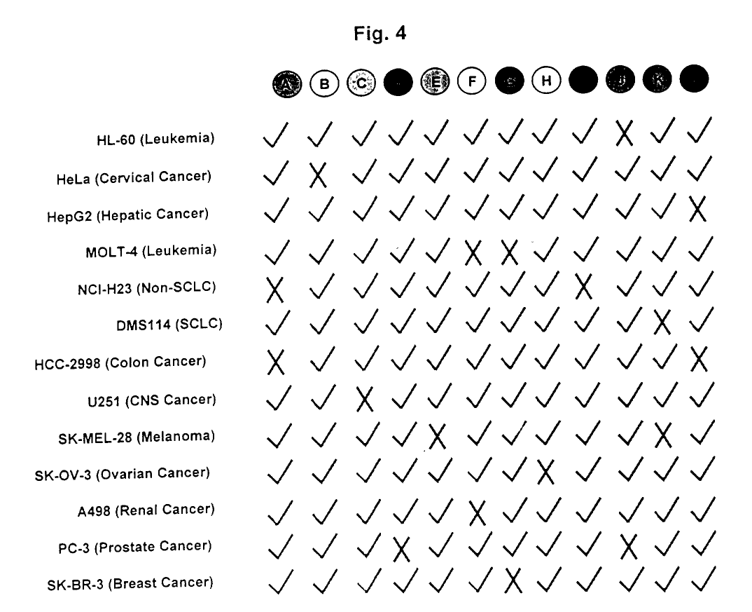

The term “cell” includes any eukaryotic cell, such as fungal cells (i.e., yeast cells), insect cells (e.g., Schneider and sF9 cells), or somatic or germ line mammalian cells, or cell lines e.g., HeLa cells (human), NIH3T3 (murine), RK13 (rabbit) cells, embryonic stem cells (e.g., D3 and J1), and cell types such as hematopoietic stem cells, myoblasts, hepatocytes, lymphocytes, and epithelial cells and, e.g., the cell lines listed as examples in FIG. 4.

The term “test cell” includes any eukaryotic cell in which the phenotypic effect of a peptide aptamer can be assessed, preferably having the ability to grow in culture (or be passaged) virtually indefinitely, i.e., an immortalized cell. In preferred embodiments, such cells are originally derived from a human cancer tissue or leukemia and can be cultivated as clonal cell lines, such as, e.g., HeLa cells or HL-60 cells. Such cells may also exhibit other art recognized hallmarks attributed to cancerous cells (e.g., lack of cell/cell contact growth inhibition; abnormal karyotype).

The term “control test cell” or “normal cell” refers to a cell (e.g., a mammalian cell) that is derived from a normal (i.e., non-diseased or healthy) tissue sample (i.e., primary cells) or cells that have been suitably altered to represent a disease-associated phenotype. In cancer-associated screens, “normal” refers to cells that typically can only be passaged in culture for a finite number of passages and/or exhibit other art recognized hallmarks attributed to normal cells (e.g., a normal karyotype). Preferably, “normal cells” are unaffected by peptide aptamers of the invention.

Overview

The phenotypic selection of aptamers expressed in mammalian cells described herein automatically identifies peptides that have the desired effect on relevant targets in an intracellular environment. Thus, the present invention has the advantage of not only facilitating and expediting the understanding of the molecular basis of cellular functions and associated diseases, but also the rapid design of therapeutics. For example, targeting of a particular protein (e.g., effector) by an aptamer in a manner that brings about a desired phenotypic change automatically validates that protein as a viable focal point for the design of effective therapeutic strategies. Secondly, the aptamer ligands themselves represent molecules upon which to base development of therapeutic small molecules (e.g., non-peptidic).

Moreover, although selectable phenotype is required, a detailed understanding of the associated pathway is not necessary. Indeed, after the fact, aptamers identified through such screens can serve as important tools for further study of pathways and functions involved in the manifestation of a selected phenotype. Of equal or greater importance, however, is the fact that the method of the invention leads to the immediate identification of novel molecules with a desired function in an intracellular context. This is in contrast to a variety of other research strategies, the goal of which is to identify novel targets, with the hope that identification of ligands with desired effects on that target, will then be possible.

The regulatory networks that govern various functions in eukaryotic cells, especially in multicellular organisms and mammals, are extremely complex. Not only is there intrinsic and extensive overlap, crosstalk and redundancy, but the extent to which present methods of analysis, especially in mammalian cells (e.g. overexpression of a component, followed by analysis of downstream effects) may have obfuscated the mapping of these pathways is a constant concern. Even though knockout or antisense elimination of a particular gene in animals or cell lines may serve this purpose in some circumstances, these approaches are not amenable to high-throughput analysis, and are limited to the elimination of known, non-essential genes.

Inhibition or loss of one component of a signaling pathway in eukaryotic cells is often compensated by mechanisms associated with the intrinsic complexity and redundancy of these signaling networks. Accordingly, the present invention is particularly suited for screens in which the aptamer manifests a gain-of-function or restoration-of-function phenotype. Aptamer screens can identify peptides that act to induce allosteric changes in an enzyme or other regulatory protein that result in its activation. In contrast, identification of aptamers that act in a manner analogous to a classically defined recessive mutation, typically requires high levels of overexpression in order to titrate out a given function or activity. As such, gain-of-function phenotypes have the added advantage of being achievable through lower intracellular aptamer concentrations than are required for manifestation of a phenotype that depends on functional inhibition.

An additional utility of the invention is the mobility and modularity of retroviral vectors for peptide aptamer expression: peptide aptamers with a defined function in one cell type can be introduced into other cell types for phenotypic analysis. This combination of diversity and flexibility is unparalleled in nature or in other man-made systems. As described in greater herein, this flexibility allows for the development of aptamer libraries as diagnostic tools. For example, a panel of vectors expressing peptide aptamers that induce apoptosis in cancer cells can be used for “functional phenotyping” of various tissues (e.g tumor biopsy tissue), in which susceptibility to a particular aptamer can have predictive value for determining the efficacy of a certain treatment regimen. Accordingly, the present invention also provides a diagnostic kit comprising a panel of peptide aptamers, or nucleic acids encoding the aptamers (e.g. as vectors), which can be used (e.g., according to instructions enclosed in the kit) to diagnose or functionally phenotype selected cells. Alternatively, the panel of peptide aptamers or nucleic acids (e.g., in the form of a kit) can be used therapeutically to treat a disease defined by a particular cell phenotype (e.g., the gain or loss of a normal function).

Generation of Peptide Aptamer Libraries

The invention can be performed using any art recognized vector system suitable for expressing short nucleic acid sequences in a eukaryotic cell. In a preferred embodiment, the invention employs high-titer retroviral packaging systems to produce peptide aptamer libraries. Various vectors, as well as amphotropic and ecotropic packaging cell lines, exist that can be used for production of high titers of retroviruses that infect mouse or human cells (Burns et al., 1993; Pear et al., 1993). These delivery and expression systems can be readily adapted for efficient infection of any mammalian cell type, and can be used to infect tens of millions of cells in one experiment. Aptamer libraries comprising nucleic acid sequences encoding random combinations of a small number of amino acid residues, e.g., 5, 6, 7 or more, but preferably less than 100, more preferably less than 50, and most preferably less than 20, can be expressed in retrovirally infected cells as free entities, or depending on the target of a given screen, as fusions to a heterologous protein, such as a protein that can act as a specific protein scaffold (for promoting, e.g., expressibility, intracellular or intracellular localization, stability, secretability, isolatablitiy, or detectability of the peptide aptamer; see below for further details). Libraries of random peptide aptamers when composed of, for example 7 amino acids, encode molecules large enough to represent significant and specific structural information, and with 10⁷ possible combinations (preferably more, e.g., 10⁹), is within a range of cell numbers that can be efficiently tested using a reasonable number of test vessels within a given phenotypic screen (e.g., to test 10⁷ or preferably, 10⁹ combinations, 100 tissue culture vessels each containing 10 mls of media with 10⁶ cells/ml can be used).

Depending upon the intended use of a library, the peptide aptamers can be expressed as fusions to different functional moieties. For example, scaffolds can be “neutral” ones that increase stability or allow monitoring of expression (e.g. a catalytic or detectable moiety such as chloramphenicol acetyltransferase, 3-galactosidase, or green fluorescent protein). Alternatively, scaffolds can be used that introduce structural constraints to the expressed peptide aptamer (e.g. presentation of the aptamer within an exposed thioredoxin loop), or that encode targeting domains such as a nuclear or membrane localization signals. In screens for peptide aptamers that induce apoptosis of cancer cells (described in detail below), functional domains from a apoptotic regulatory proteins, such as the art recognized BH3, DD, BIR3, or BIR domains, can be used to direct peptide aptamers to cellular apoptotic machinery or regulatory circuits. In other embodiments, aptamer libraries can also be generated that fuse other functional entities to the aptamer, such as transcriptional activation or repression domains, a CAAX farnesylation signal sequence that directs membrane localization, MHC proteins, SH2, or SH3 domains.

As described in detail herein, the peptide aptamer libraries can be generic (i.e., encode only minimal, random peptide aptamers), or incorporate features that suit them for the study of particular phenotypes associated with certain processes or specific intracellular locations (e.g., a heterologous localization domain).

Aptamer Library Screening, Validation, and Analysis

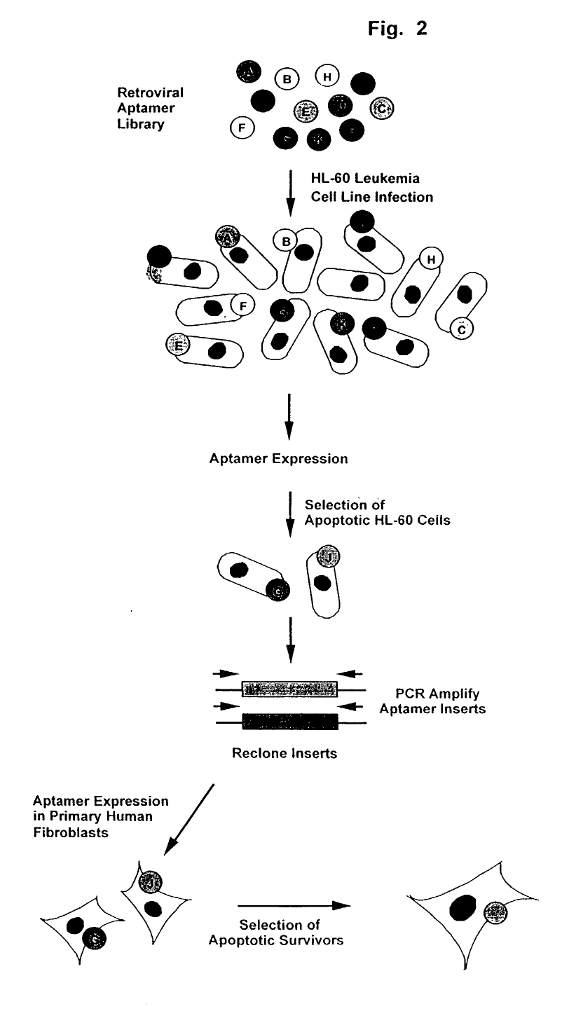

In practicing the invention, a population of cells, preferably a clonal population of eukaryotic cells is infected, and cells with a desired phenotype, or a phenotype which differs from other cells in the cell population, is selected or isolated. Coding sequences of aptamers selected in the first round of screening can be amplified by PCR, re-cloned, and re-introduced into naïve cells. Phenotypic selection can then be repeated in order to validate individual aptamers within the original pool. Aptamer coding sequences within cells identified in subsequent rounds of selection can be iteratively amplified and subcloned and the sequences of active aptamers can then be determined by DNA sequencing using standard techniques. A schematic of this iterative process is shown in FIG. 2. This strategy can be applied to the identification of aptamers associated with a wide variety of cellular processes including, e.g., cell proliferation, regulation of apoptosis, protein trafficking or transport, cell motility or differentiation, and modulation of various signal transduction networks.

In a particular embodiment, the invention is used for the identification of aptamers that modulate apoptosis, e.g., induce apoptosis in cancer cells. Accordingly, it is understood that uses of these particular aptamers includes therapeutic, diagnostic, and research applications.

Identification and Uses of Aptamers that Induce Apoptosis in Cancer Cells

There are more than 100 types of cancer, with subtypes and phenotypically distinct subsets being continuously defined. It is not clear how many individual metabolic or epigenetic changes are necessary for cancer to occur, although several alterations in cell physiology are thought to be required for cells to become cancerous (reviewed in Boland and Ricciardiello, 1999). Among these are the perpetuation of growth signals, limitless lifespan, as well as metabolic changes that mediate angiogenesis, tissue invasion, and metastasis. Associated with an alteration of growth and replication signals, is a related requirement: cancer cells must evade apoptotic signals that would normally arise due to inappropriate signals for cell division. (reviewed in Hanrahan and Weinberg, 2000).

Apoptotic death can be triggered by a wide variety of stimuli. Hundreds of different agents have been identified that induce apoptosis in various cancer cell lines. However, not all cells necessarily respond to the same stimuli; there appear to be both multiple apoptotic signaling systems, as well as multiple pathways that mediate evasion of these signals. The process of unraveling the mechanistic details of apoptosis has revealed a core machinery involved in the execution of later steps (i.e. caspase proteolytic cascades), as well as multiple regulatory networks that govern the apoptotic response. These regulatory pathways frequently respond to environmental signals such as growth signaling or stress, and subsequently feed into the core apoptotic machinery at a variety of positions. A common feature of these signaling pathways that impinge on this machinery is that nearly every level is paired with a counteracting anti-apoptotic signal mediator. The life-and-death decision of a particular cell is a precarious balance between these forces.

To selectively exploit this precarious balance that dictates survival of a cancer cell, a retroviral aptamer expression library containing short peptides can be used to infect clonal cancer cell lines, followed by selection of apoptotic cells. In this screen, it is important to discriminate between spontaneous apoptosis and aptamer induced apoptosis by inducing aptamer expression at a given time, and identifying the cells that have undergone apoptosis subsequent to this induction. Although a number of transcriptional regulation systems exist that regulate transcription upon exposure or removal of specific compounds (e.g. ecdysone or tetracycline), these systems require multiple time consuming modifications of the host cell, including integration and stable expression of several plasmids harboring the numerous components of the system. Expression of these various required components can be unstable and unreliable.

As an alternative approach to achieving the goal of tightly regulated aptamer expression in the apoptotic aptamer screen, expression can be regulated by adapting a method described by Werstuck and Green (1994). Briefly, a retroviral vector library containing nucleic acid sequence encoding random peptide aptamers is engineered where the random peptide aptamers are under the constitutive control of a strong promoter (such as, e.g., the CMV or SV40 promoter), but translation is suppressed through association of a leader sequence with an RNA binding agent, e.g., a cell permeable dye, that binds to a portion of the transcript and prevents translation and expression of the downstream peptide. This approach has the distinct advantage of not requiring de novo preparation of different host cells for every screen, since this regulatory feature is incorporated into the library itself.

Accordingly, a retroviral peptide aptamer library can be used to transduce ten to 100 million cells growing in suspension. For example, HL-60 cells can be used since they have robust growth in suspension, and have well characterized responses to various apoptotic stimuli, which can be useful in examining the apoptotic phenotypes associated with various aptamers. Aptamer dependent apoptosis can be distinguished from spontaneous apoptosis by presorting and removing cells undergoing apoptosis prior to aptamer expression. Cells are then washed to remove the RNA binding agent, allowing translation of the aptamer peptide. Cells are then re-sorted to identify cells in which apoptosis occurred following aptamer expression.

In the initial screening steps, apoptotic cells are identified using APOPTEST™ or an analogous method, both before and after induction of aptamer expression. Briefly, in contrast to TUNEL staining methods for identifying apoptotic cells, which identify apoptosis by end-labeling DNA fragments that arise late, APOPTEST™ stains cells early in apoptosis, and does not require fixation of the cell sample (Martin et al., 1994). This method utilizes an annexin V antibody to detect cell membrane re-configuration that is characteristic of cells undergoing apoptosis. Apoptotic cells stained in this manner can then sorted either by fluorescence activated cell sorting (FACS), or by adhesion and panning using immobilized annexin V antibodies.

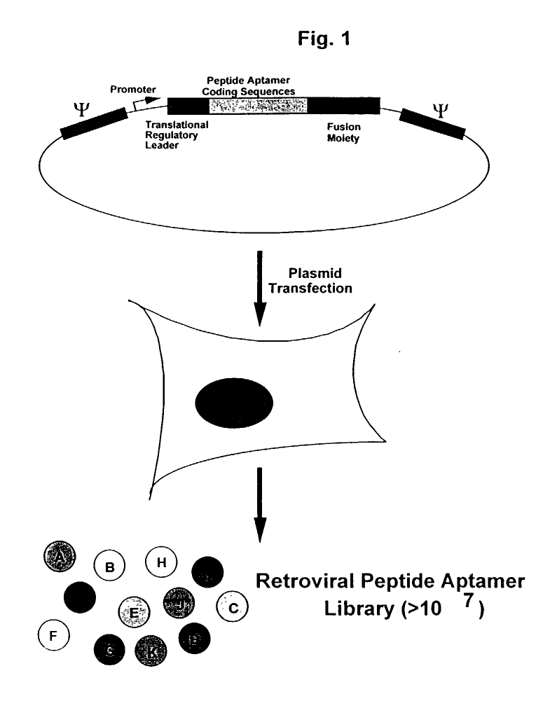

Retroviral sequences in cells identified and segregated in this manner can be amplified by PCR, and the aptamers can be recloned and validated as described in FIG. 1. In later rounds of aptamer re-screening and validation, at the point where aptamers are being re-tested individually, other methods of apoptosis can be employed as a counterscreen. These include such methods as TUNEL staining or propidium iodide staining. This is necessary to ensure that the selected phenotype is in fact apoptosis, and not an aptamer-induced alteration in membrane metabolism. Validated apoptotic aptamers are then expressed in a variety of non-cancerous cells and other cancer cell lines to determine their specificity and range of action. Aptamers can be identified that do not induce apoptosis in non-tumor cells, although they can also be evaluated for their ability to induce apoptosis in other cell lines, as described herein.

The invention also encompasses screens that can be conducted for identification of aptamers that augment the sensitivity of cancer cells to radiation or cancer chemotherapeutic agents. In addition, aptamers can act synergistically with the apoptotic response to these agents, either by impacting the same pathways, or by targeting novel but parallel cellular responses to these agents. In each case, aptamer library expression can be induced in a population of transduced cells, followed by treatment with an agent known to induce apoptosis, but at a dose below the threshold for this response.

Phenotypic Analysis of Tumor Cells: Susceptibility to Peptide Aptamer Mediated Apoptosis

Upon identification of aptamers that induce apoptosis in a clonal cancer cell line, but not in normal cells, a retrovirus can be constructed for expression of each aptamer. Translational control sequences would not be necessary in these library constructs. Use of a retroviral vector at this step has the advantage of allowing expression in any cell type, whereas use of a plasmid at this step would limit its use to cells into which it can be efficiently transfected. This aptamer expressing retrovirus can then be used to infect and express a given aptamer in a panel of clonal cancer cell lines, such as those in the DTP Human Tumor Cell Line Screen (Monks et al., 1991), which represents a diverse set of clonal cancer cells derived from various types of human tumors. The susceptibility of each cell type to apoptosis when infected by a retrovirus encoding the first aptamer can be documented.

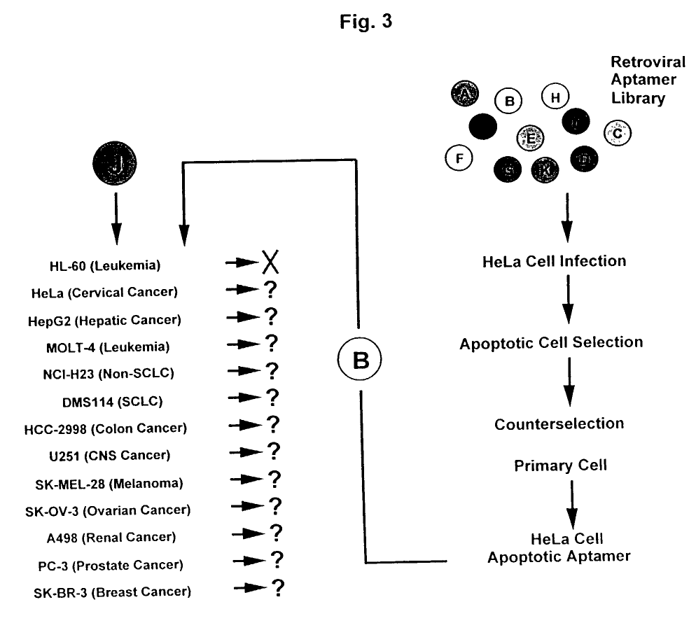

The library screening process can then be repeated in a second cell line in which expression of the first aptamer did not induce apoptosis. Aptamers identified in screening of this second cell line would also be tested against the entire cell line panel (shown in FIG. 3). This iterative process of identifying an aptamer with activity in one cell line, determining its activity against other cell lines, followed by identification of additional aptamers active against other cell lines, eventually leads to coverage of the entire cell line panel: a set of aptamers that induces apoptosis in at least one type of cancer cell line in the panel. An idealized compilation of the outcome of this process is shown in FIG. 4. It is important to reiterate that the pattern of susceptibility of a cancer cell to the identified set of aptamers can serve as a phenotype in and of itself, since this susceptibility can indicate the manifestation of the aggregate of changes that the cell underwent in its pathogenesis. Thus, this operational categorization of cancer cells can be extremely valuable, even in the absence of a full understanding of the molecular basis of the action of a given aptamer.

Profiling “Aptamer Susceptibility” of Cancer Cells

The invention provides the ability to generate an apoptotic aptamer phenotype, i.e., a profile of aptamers that induces apoptosis and therefor represents important information about a given tumor cell. It allows categorization of any clonal population of tumor cells with respect to the most pertinent and important type of information: how to destroy with selectivity and specificity. In a clinical setting, for example, profiling of aptamer-associated apoptosis, conducted using, e.g., the kits containing panels of aptamers described herein, can be used by investigators to draw correlations between the aptamer apoptosis phenotype and clinical prognosis, or serve as a predictive tool for the effectiveness of a given therapeutic strategy.

Induction or Inhibition of Apoptosis in Other Cell Types

Aptamers can be identified that induce apoptosis in cells associated with other hyperproliferative disorders. These include, for example, prostatic hyperplasia in aging men and psoriasis. In addition, apoptosis is associated with diseases like osteoporosis, in which induction of osteoclast apoptosis-cells that resorb bone, is of potential therapeutic benefit (Rezka et al., 1999). Conversely, aptamer screens can also be performed to identify inhibitors of osteoblast apoptosis. The therapeutic benefit of inhibition of apoptosis in the bone for generating needed cells is a desirable result (Plotkin et al., 1999).

Other Advantages of the Aptamer System

Another advantage of the present invention is integration of the primary screen with effective counter-screens that demonstrate the specificity of the phenotype. For example, cancer cells frequently undergo epigenetic changes that allow them to ignore normal growth regulatory signals, including apoptosis. In searching for aptamers that induce apoptosis in a given type of tumor cell, it will be important to make sure that these aptamers do not induce apoptosis in normal cells. In other examples, where an aptamer might be identified that modulates trafficking or transport of a particular protein, the invention provides the ability to incorporate counterscreens to determine that aptamer induced changes in localization are relatively specific to the phenotype of interest.

The present invention also allows for identifying unlikely events such as, that one aptamer is identified that induces apoptosis in all cancer cells, but not in primary cells. Accordingly, such a result indicates that it would be superfluous to carry out subsequent rounds of library screening for this phenotype, but that the aptamer is a lead candidate to be tested for therapeutic activity. In addition, such a candidate aptamer can be tested for its ability to potentiate conventional cytotoxic cancer chemotherapeutics. Accordingly, the apoptosis screens described herein can also be carried out in the presence of sub-cytotoxic concentrations of these agents, in order to identify aptamers that can serve as an adjunct to lower the dose at which a given chemotherapeutic compound is effective, thereby lowering the toxicity and side effects suffered by treated patients.

Diagnostic Use

The methods and compositions of the invention can also be used for diagnostic purposes. Accordingly, the retroviral aptamer libraries disclosed herein, or aptamers encoded by these libraries, can be packaged into kits with instructions for use. These kits can be used to screen for desirable aptamers using a format described herein for phenotyping, e.g., a cancer cell or tissue derived from, e.g., a biopsy sample. For example, a panel of vectors expressing peptide aptamers that induce apoptosis in cancer cells can be used for “functional phenotyping” of tumor biopsy tissue, in which susceptibility to a particular aptamer can have predictive value for determining the efficacy of a certain treatment regimen. Alternatively, the kit may be used in conjunction with the cancer cell lines disclosed herein, other art recognized cell lines, or a combination thereof. In addition, the aptamer libraries may be used in conjunction with other screening technology involving, e.g., phage display and/or yeast two-hybrid systems for testing or validating a given aptamer. Still further, the methods and compositions described herein may also be used in conjunction with various art recognized gene chip technologies to, e.g., phenotype or diagnose a cancer. For example, the aptamer approach can be combined with gene chip technologies in order to enable the high-throughput quantitation of the expression of thousands of genes in a sample. This combined approach can be applied to the study of, e.g., diffuse large B-cell lymphoma (DLBCL), the most common subtype of non-Hodgkin's lymphoma, in order to discover identifiable differences in aptamer susceptibility and also gene expression patterns that correlate with and distinguish tumor proliferation rate, host response, and differentiation state of the tumor (Alizadeh et al., 2000). Any of the forgoing composite diagnostic approaches are understood to be within the scope of the invention.

Gene Therapy

The therapeutic peptide aptamers capable of modulating a cell phenotype can be delivered to cells by methods of gene therapy. Following testing of a cancer biopsy sample for susceptibility, a vector encoding a predetermined aptamer can be injected directly into the tumor, or delivered in any other art-recognized manner of gene therapy. An advantage of the invention is that treatment of a given cancer in a subject with a vector encoding a therapeutic aptamer is an acute undertaking, which does not require perpetual expression of the introduced gene, which has been a difficulty in most other gene therapy approaches (Verma and Somia, 1997). In one approach, aptamer expressing viruses are used either as stand-alone therapeutics, or as adjuncts to other therapeutic regimens. For example, retroviruses can be injected directly into solid tumor sites to minimize the possibility of side-effects. In addition, aptamers can be identified that act in concert with other cancer therapeutic drugs or radiation therapy in a manner that lowers their effective doses, thereby decreasing toxicity or side effects of these treatments. Indeed, library screens can be undertaken in which aptamer expression results in apoptosis of a cell line in the presence of a sub-apoptosis inducing concentration of a conventional cancer therapeutic agent (e.g. tamoxifen or camptothecin).

Use of Aptamers for the Research and Development of Other Therapeutics

Elucidation of aptamer targets can also serve as a powerful tool for the discovery of novel cellular targets that advance our understanding of the impacted cellular pathway/s. As such, the invention also encompasses retroviral aptamer libraries (e.g., in the form of kits), for use by basic researchers for genetic exploration of complex pathways in mammalian cells.

Accordingly, the invention can be used for the molecular classification of tumors and identification of previously undetected and clinically significant subtypes of cancer. In addition, the invention can be used as therapies that regulate or manage tumor growth (Balis, 1998).

Another embodiment of the invention includes the use of the aptamers as lead molecules for drug development. For example, using any art recognized molecular modeling techniques, an aptamer can be used for designing and synthesizing other molecules having the desirable function of the aptamer but also having other desirable traits such as cell solubility, potency, time-release properties, etc.

Other features of the invention will be apparent from the following examples which should not be construed as limiting.

Identifying Peptide Aptamers Capable of Altering a Cell Phenotype Using Transgenic Animals

The methods of the invention can also be used to identify peptide aptamers capable of altering a cell phenotype in a non-human transgenic or gene-knockout animal. For example, in one embodiment, a library of peptide aptamers encoded in an eukaryotic expression vector, e.g., a retroviral vector can be introduced into a transgenic animal having a detectable phenotype. The detectable phenotype may be a visually or molecularly recognizable phenotype and includes, for example, an alteration in the growth, maintenance, migration, or function of a cell type or tissue of the animal. Transgenic animals suitable for introducing an aptamer library include animals engineered to have, e.g., a cancer (e.g., an animal having a constitutive promoter driving the expression of an oncogene or, alternatively, an animal engineered to lack a tumor suppressor) thereby allowing for the screening of aptamers which can abrogate a cancer phenotype. Alternatively, animals engineered to have a gene disruption, i.e., a transgenic “knock-out” animal, can be used to screen the aptamer library for peptide aptamers that can rescue the function normally provided by the disrupted gene. Using either of the foregoing strategies, peptide aptamers that can affect a “gain of function” or loss of function” can be screened or selected for in vivo. Then, the cells or tissue from an animal exhibiting the desired phenotype is then used as a source of biological material for the isolation and identification of the nucleic acid encoding the peptide aptamer associated with the phenotype using art recognized techniques.

The invention also encompasses the ex vivo treatment of cells, e.g., cells derived from one of the transgenic animal described above, with a peptide aptamer library. The treated cells can then be studied in vitro or introduced into a host animal and monitored using art recognized techniques. For example, desired cell types or tissues that can be treated ex vivo and then reintroduced into a host animal following exposure to an aptamer library include, cells of the nervous system, muscle cells, and hematopoietic cells. In a preferred embodiment, hematopoietic cells lacking an gene needed for normal blood cell development or function, for example, a growth factor or a receptor, e.g., a T cell receptor, are contacted with an aptamer library and then introduced into a host animal, for example a host animal that has been treated so as to lack its normal blood cell repertoire (using, e.g., radiation). The animals treated with the cells exposed to the aptamer library are then monitored for the appearance of a desired phenotype (e.g., the repopulation of a particular blood compartment or outgrowth of a certain cell type), and such cells can then be isolated and used as a source of material for identifying an aptamer associated with the phenotype. Using the foregoing approach, the invention is suitable for screening peptide aptamers that are capable of affecting, e.g., cancers of the blood (e.g., mechanisms of leukemogenesis), immune cell function (e.g., T cell receptor function and/or other immune cell interactions), and various other diseases of the blood (e.g., hemochromatosis, or viral infections, e.g., an HIV infection).

As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. Methods for generating such transgenic animals (e.g., via embryo manipulation and microinjection), particularly animals such as mice, are well known in the art as described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, Second Edition (Cold Spring Harbor Laboratory Press, Cold Spring Harbor, N.Y., 1994).

### EXEMPLIFICATION

Throughout the examples, unless otherwise indicated, the practice of the present invention will employ conventional techniques of chemistry, molecular biology, microbiology, recombinant DNA technology, cell culture, and animal husbandry, which are within the skill of the art and are explained fully in the literature. See, e.g., Sambrook, Fritsch and Maniatis, Molecular Cloning: Cold Spring Harbor Laboratory Press (1989); DNA Cloning, Vols. 1 and 2, (D. N. Glover, Ed. 1985); Oligonucleotide Synthesis (M. J. Gait, Ed. 1984); Nucleic Acid Hybridization (B. D. Hames and S. J. Higgins, Eds. 1984); the series Methods In Enzymology (Academic Press, Inc.), particularly Vol. 154 and Vol. 155 (Wu and Grossman, Eds.; Large-Scale Mammalian Cell Culture Technology, Lubiniecki, A., Ed., Marcel Dekker, Pub., (1990); Molecular and Cell Biology of Yeasts, Yarranton et al., Ed., Van Nostrand Reinhold, Pub., (1989); Yeast Physiology and Biotechnology, Walker, G., John Wiley & Sons, Pub., (1998); Baculovirus Expression Protocols, Richardson, C., Ed., Humana Press, Pub., (1998); Methods in Plant Molecular Biology: A Laboratory Course Manual, Maliga, P., Ed., C. S. H. L. Press, Pub., (1995); and Current Protocols in Molecular Biology, eds. Ausubel et al, John Wiley & Sons (1992)).

### EXAMPLE 1

### Methods for Screening Peptide Aptamers Capable of Modulating Apoptosis in a Human Myeloid Leukemia

In this example, methods for screening peptide aptamers capable of modulating apoptosis in human myeloid leukemia HL-60 cells are described.

HL-60 cells are a well characterized human myeloid leukemia cell line in which apoptosis is inducible. These cells also grow in suspension, and their apoptotic response to multiple stimuli has been characterized (reviewed in Darzynkiewicz et al., 1992). Accordingly, in one approach, cells pre-cleared of spontaneously apoptotic cells are contacted with an aptamer library encoded in an expressible form on a plasmid, preferably a retrovirally derived vector, that can efficiently enter the cell and express a particular aptamer. Then, HL-60 cells in which apoptosis has been induced by an expressed aptamer, are identified. Any art recognized FACS or panning strategies can be used for detecting the approximate 100-1000 apoptotic cells per 10 million cells that represent a desirable level of sensitivity and selectivity required for the apoptotic aptamer screen.

After selection of apoptotic cells is achieved, the aptamers that induce apoptosis in HL-60 cells, are then tested for their ability cause apoptosis in non-cancerous human cells, such as primary fibroblasts, with a preferably result being that the selected aptamer works preferentially in only cancerous cells.

Each aptamer identified in the HL-60 screen is then tested in a diverse panel of human cancer cell lines. This aspect of the invention allows for the identification of a set of aptamers sufficient to induce apoptosis in as many different types of cancer cells as possible.

### EXAMPLE 2

### Methods for Screening Peptide Aptamers Capable of Modulating Apoptosis Using Growth Factor Dependent Cells

In this example, methods for screening peptide aptamers capable of modulating apoptosis using BaF3 cells are described.

To identify and catalog aptamers that cause apoptosis of cancer cells, a well established growth factor dependent (IL-3) BaF3 cell-based assay system can be employed (see, e.g., Kitamura et al., 1995). This system affords the identification of aptamers that induce activation of the erythropoietin mediated signal cascade in hematopoietic stem cells in the absence of growth factor. Specifically, the cells are used to first screen and identify retrovirus encoded peptide aptamers that abrogate apoptosis of BaF3 cells in response to withdrawal of erythropoietin.

In this screen, a retroviral library is used to infect mouse BaF3 cells, which normally undergo apoptosis upon withdrawal of IL-3. However, this apoptotic phenotype is not observed in the presence of activated forms of the abl oncogene. Thus, expression of aptamers that stimulate Abl kinase activity, or the activity of appropriate downstream components that signal transduction pathway, can result in cell survival. Aptamer coding sequences from the surviving cells are then amplified by PCR, recloned into a mammalian expression vector, and re-screened by reintroduction into naive BaF3 cells.

This assay system allows for the discovery of active aptamers, and phenotypes associated with aptamer expression can be easily deconvoluted in this system. For example, aptamers that cause survival of BaF3 cells through stimulation of Abl can be identified by the susceptibility of these cells to Novartis ST1571, a specific inhibitor of the Abl kinase that is in clinical use for the treatment of some leukemias (Carroll et al., 1997). The survival phenotype of these cells can the be reversed by the ST1571 kinase inhibitor, whereas aptamer mediators of Jak2 or STAT5 activity can be identified by examination of these proteins and their activities in aptamer expressing cells (Nosaka et al., 1999; McCubrey et al., 2000). Aptamers can then be expressed ectopically in erythropoietin receptor knockout mice. These mice are deficient for erythropoiesis, which is reconstituted by activation of the Abl kinase (Ghaffari et al., 1999).

In addition, aptamers that specifically substitute for erythropoietic signals can be used as lead compounds for the development of small molecule drugs for therapeutic use in the treatment of anemia in kidney dialysis and cytotoxic chemotherapeutic treatments.

Further, if desired, aptamers identified in the BaF3 anti-apoptotic screen can also be tested for their effects on hematopoietic stem cell development and differentiation.

Accordingly, this system allows for the identification of active aptamers capable of modulating apoptosis and a method for understanding their mode of action.

### EXAMPLE 3

### Methods for Screening Peptide Aptamers Capable of Modulating Intracellular Signaling Cascades

In this example, methods for screening peptide aptamers capable of modulating intracellular signaling cascades are described.

In general, signaling cascades refer to networks of molecular interactions and activities through which an environmental or developmental stimulus is received and interpreted by a cell. This carefully orchestrated molecular response is a designated sequence of events that ultimately leads to an alteration in cellular metabolism or function. G protein coupled receptors (GPCRs) are a large and growing gene family of transmembrane proteins. To date, over 1000 GPCRs have been cloned. These receptors are classified both by the types of extracellular signals to which they respond (e.g. photons, odors, ions, monoamines, or peptides), and by the particular trimeric G protein effector complex that mediates intracellular transmission and amplification of receptor signaling. Ligand mediated signaling through these receptors results in a broad spectrum of responses.

The present invention provides aptamer libraries that can be screened for members that modulate a cellular response analogous to that resulting from ligand engagement by a given receptor, or that inhibit such a response, in the presence of ligand. The invention also provides methods for screening libraries to identify aptamers that abrogate, attenuate, or alter the specificity of receptor mediated signaling that occurs upon binding of the receptor by a cognate (endogenous or exogenous) ligand. Receptor tyrosine kinase signaling cascades, as well as receptor mediated signaling cascades that mediate signaling through src family kinases, are other pathways that can also be targeted using this system.

### EXAMPLE 4

### Methods for Screening Peptide Aptamers Capable of Modulating Protein Transport and Trafficking

In this example, methods for screening peptide aptamers capable of modulating protein transport and trafficking are described.

The present invention provides methods for identifying aptamers that can affect trafficking of specific proteins to the cell surface. This has particular utility in cases where misdirection of proteins is associated with disease. Aptamer libraries can be introduced into clonal cell lines stably expressing the mislocalized protein. Cells expressing aptamers affect membrane localization of the desired protein can be identified by staining non-permeabilized cells with a specifically reactive antibody. Positively staining cells can then be physically separated by either fluorescence activated cell sorting (FACS) or other appropriate art recognized techniques. Aptamers can be identified that correct the mislocalization or induce the relocalization of any protein at the cell surface, including various receptors and channels, antigens, or proteins involved in the immune response. In the latter case, involving modulation of antigen presentation in an immune response, an aptamer can either augment an immune response to specific infections, especially in immunocompromised individuals, or attenuate certain aspects of immunity that can be beneficial in autoimmune syndromes or conditions.

An example of a particular application of the foregoing methods can be for the development of therapeutics for hemochromatosis, an autosomal recessive disorder in which approximately 95% of the non-functional protein encoded by mutant alleles is no longer directed to the cell surface (Waheed et al., 1997). This leads to an alteration of iron transport in certain intestinal cells of individuals homozygous for this mutant allele, and chronic accumulation of iron in the serum to levels that lead to long-term organ toxicity. Hemochromatosis is, in fact, the most common hereditary disorder among Caucasians, affecting up to one in every two hundred Americans, and leading to liver, kidney, and other organ failure, the etiology of which, had not been previously appreciated.

Still another application of the foregoing methods is the following. Many viruses, including HIV infected T cells in which the HIV nef gene product down-regulates MHC-mediated antigen presentation (reviewed in Collins and Baltimore, 1999), various herpesviruses, including cytomegalovirus (CMV) (del Val et al., 1997; Kleijnen et al., 1997), and papillomavirus, actively suppress antigen presentation as a means of eluding or evading immune recognition and response (reviewed in McFadden and Kane, 1994). Clonal cell lines either chronically infected by these viruses, or constitutively expressing virus encoded proteins that affect these functions involving protein transport or trafficking, can be infected with a retroviral aptamer library, and cells in which antigen presentation was augmented or reconstituted as measured by, e.g., FACS, can be scored using the subsequent steps described above, and a candidate aptamer that can modulate the pathway can be identified.

### EXAMPLE 5

### Methods for Screening Peptide Aptamers Capable of Modulating Cell Adhesion

In this example, methods for screening peptide aptamers capable of modulating cell adhesion are described.

Cell adhesion is an important element of development and the immune response. Cell surface adhesion molecules function both as mediators of physical association between cells and as important sensors and transmitters of intracellular signals. For example, the integrin proteins of leukocytes and neutrophils serve as adhesive molecules that immobilize these cells to sites of localized immune response, and in turn, trigger intracellular responses upon adherence (i.e. degranulation). The cell sorting protocols described herein (e.g., Example 1) can be easily adapted for panning, in which aptamers that can modulate (e.g., induce certain adhesive properties in a cell) are identified.

### EXAMPLE 6

### Methods for Screening Peptide Aptamers Capable of Modulating Membrane Transport

In this example, methods for screening peptide aptamers capable of modulating membrane transport are described.

Membrane transport of ions and other ligands plays an important role in many physiological processes and disease states. For example, ATP cassette transport proteins have a wide variety of functions, including mediating efflux of drugs. The human multiple drug receptor membrane protein (MDR) presents a significant clinical problem in patients undergoing cancer chemotherapy, by efficiently pumping cancer therapy drugs out of cancer cells, thereby limiting their efficacy. Other members of this family are associated with peroxisomes, mutant forms of which are associated with disease, including adenoleukodystrophy. Art recognized dyes exist that can be used to identify cells in which these transporters are unable to mediate efflux of certain types of compounds.

Accordingly, these techniques can be used to screen retroviral peptide aptamer libraries when used to infect clonal cell lines that endogenously overexpress an MDR or other ATP cassette protein, or in which this gene or a mutant form is stably expressed. Cells expressing aptamers capable of increasing dye retention in these cells can be sorted, and the sequence of the encoded aptamer can be determined.

### EXAMPLE 7

### Methods for Screening Peptide Aptamers Capable of Modulating Cell Motility and Chemotaxis

In this example, methods for screening peptide aptamers capable of modulating cell motility and chemotaxis are described.

Neutrophils are among the first leukocytic cells to migrate into tissues in response to invading pathogens or other initiators of inflammatory injury. One of the first steps of neutrophil involvement in acute inflammation is chemotaxis, directed movement toward chemotactic agents, such as complement fragments (C5a), cytokines (IL-8), leukotrienes, and bacteria-derived peptides such as formyl-methionine-leucinephenylalanine (fMLP). Inhibition of this chemoattractive response is an effective means of abrogating inflammation, especially in diseases like asthma and the chronic inflammation associated with cystic fibrosis.

Accordingly, cell lines stably expressing a chemotactic receptor can be infected with a retroviral peptide aptamer library, and migration toward a specific chemoattractant can be measured using art recognized transwell assays in which the cells are placed in an upper chamber, and the chemoattractant is placed in a lower chamber. After a time sufficient for transmigration of the chemotactic cells across the chamber barrier, cells remaining in the upper chambers can be pooled, grown out, and re-assayed serially until a population of truly non-responsive cells is identified.

Thus, using this approach, which can be readily adapted to a high throughput format, aptamers that can modulate cell motility and/or chemotaxis can be identified.

### PUBLICATIONS REFERRED TO IN THE SPECIFICATION

Alizadeh et al., Nature 403:503-511 (2000)

Aravind, et al., Trends Biochem Sci. 24:47-53 (1999)

Balis, Oncologist 3:V (1998)

Baserga, Cell 79:927-930 (1994)

Blind, Proc. Natl. Acad. Sci. USA. 96: 3606-3610 (1999)

Boland, et al., Proc Natl Acad Sci USA. 96:14675-14677 (1999)

Burns et al., Proc Natl Acad Sci USA. 90: 8033-8037 (1993)

Carroll et al., Blood 90:4947-52 (1997)

Cohen et al., Proc. Natl. Acad. Sci. USA. 95:14272-14277 (1998)

Colas et al., Nature 380:548-550 (1996)

Collins et al., Immunol. Rev. 168:65-74 (1999)

Curt, Oncologist 1: II-III (1996)

Darzynkiewicz et al., Cytometry 13:795-808 (1992)

del Val et al., J. Exp. Med. 76:729-38 (1992)

Evan et al., Cell 69:119-128 (1994)

Fisher et al., Science 286:2507-2510 (1999)

Gerszten et al., Proc. Natl. Acad. Sci. USA. 96:8567-8572 (1999)

Ghaffari et al., Proc. Natl. Acad. Sci. USA. 96:13186-131901(1999)

Hanrahan et al., Cell 100:57-70 (2000).

Harrington et al., Curr. Opin. Genet. Dev. 4:120-129 (1994).

Ikushima et al., Proc Natl Acad Sci USA. 94:2609-2614 (1997).

Kitamura et al., Proc Natl Acad Sci USA. 92:9146-9150 (1995).

Klausner et al., AACR-NCI-EORTC International Conference. Washington, D.C., (Nov. 16, 1999)

Kolonin et al., Proc. Natl. Acad. Sci. USA. 95:14266-14271 (1998).

Li et al., Curr. Opin. Cell Biol. 11:261-266 (1999)

Martin et al., J. Exp. Med. 182:1545-1556 (1994)

McCubrey et al., Leukemia 14:9-21 (2000)

McFadden et al., Adv. Cancer Res. 63: 117-209 (1994)

McLafferty et al., Gene 128:29-36 (1993)

Monks et al., J. Nat. Cancer Inst. 83:757-766 (1991)

Norman et al., Science 285:591-595 (1999)

Nosaka et al., EMBO J. 18:4754-4765 (1999)

Osborne et al., Curr. Opin. Chem. Biol. 1:5-9 (1997)

Pear et al., Proc Natl Acad Sci USA. 90:8392-8396 (1993)

Polyak et al., Nature 389:300-305 (1997)

Schlessinger, Cell 100:293-296 (2000)

Schlesinger et al., Frontiers in Bioscience 3:1181-1186 (1998)

Sellers et al., Journal Clin. Invest. 104:1655-1661 (1999)

Shaw et al., Proc Natl Acad Sci USA. 89: 4495-9 (1992)

Shi et al., Proc. Natl. Acad. Sci. USA. 96:10033-10038 (1999)

Shih et al., Cell 29:161-169 (1982)

Tan et al., J. Am. Chem Soc. 120:8565-8566 (1998)

Tao et al., Proc. Natl. Acad. Sci. USA. 97:783-786 (2000)

Yonish-Rouach et al., Nature 352:345-7 (1991)

Verma et al., Nature 389:239-242 (1997)

Vojtek et al., J. Biol. Chem. 273:19925-19928 (1998)

Waheed et al., Proc. Natl. Acad. Sci. USA. 94: 12384-12389 (1997)

Werstuck et al, Science 282:296-298 (1998)

Zujewski et al., J. Clin. Oncol. 18:927 (2000)

Equivalents

Those skilled in the art will recognize, or be able to ascertain using no more than routine experimentation, many equivalents to the specific embodiments of the invention described herein. Such equivalents are intended to be encompassed by the following claims.

What is claimed:

1. A method of identifying a peptide aptamer capable of modifying a cell phenotype comprising:
a) contacting cells with a library of expressible nucleic acid sequences encoding random peptide aptamers;
b) selecting at least one cell having an altered phenotype compared to the phenotype of the cell prior to the contacting step (a); and
c) identifying one or more peptide aptamers expressed in the selected cell.

2. The method of claim 1 further comprising amplifying the nucleic acid sequences encoding the one or more peptide aptamers identified in step c) and repeating steps a)-c) using the amplified nucleic sequences as the library of expressible nucleic acid sequences specified in step a).

3. The method of claim 2, wherein steps a)-c) are repeated two or more times.

4. The method of claim 1, wherein the altered phenotype is associated with a change in levels of apoptosis, signal transduction, protein trafficking, cell adhesion, membrane transport, cell motility, or differentiation.

5. The method of claim 1, wherein the selecting is performed by measuring a change in levels of apoptosis, signal transduction, protein trafficking, cell adhesion, membrane transport, cell motility, or differentiation.

6. The method of claim 5, wherein the change in levels of apoptosis of a cell is measured using immunohistochemistry.

7. The method of claim 5, wherein the selecting is performed by measuring a change in levels of signal transduction in a cell.

8. The method of claim 7, wherein the change in levels of signal transduction is primarily mediated by a tyrosine kinase or effectors of a tyrosine kinase.

9. The method of claim 7, wherein the change in levels of signal transduction is primarily mediated by a G protein coupled receptor or effectors of a G protein coupled receptor.

10. The method of claim 1, wherein the cells are selected from the group consisting of fungal cells, insect cells, and mammalian cells.

11. The method of claim 10, wherein the fungal cells are yeast cells.

12. The method of claim 10, wherein the mammalian cells are human cells.

13. The method of claim 10, wherein the mammalian cells are clonal cancer cells.

14. The method of claim 1, wherein said library of expressible nucleic acid sequences are encoded in a eukaryotic expression vector.

15. The method of claim 14, wherein the eukaryotic expression vector is a retroviral vector.

16. The method of claim 1, wherein said peptide aptamer comprises 5 to 9 amino acid residues.

17. The method of claim 16, wherein said peptide aptamer is fused to an additional amino acid sequence selected from the group consisting of thioredoxin, a regulatory polypeptide involved in apoptosis, bcl-2, p53, an NFKB-related polypeptide, a caspase, PTEN, myc, a BH3 domain, a death domain (DD), a BIR3 domain, a BIR domain, a nuclear localization signal sequence, a membrane localization signal sequence, a farnesylation signal sequence, a transcriptional activation domain, a transcriptional repression domain, and fragments thereof.

18. The method of claim 2, wherein the amplifying of the nucleic acid sequences is performed by polymerase chain reaction (PCR).

19. A peptide aptamer, derivative thereof, or corresponding nucleic acid, identified according to the method of claim 1.

20. Use of a peptide aptamer, derivative thereof, or corresponding nucleic acid, identified according to the method of claim 1 for the molecular modeling of an agent having similar binding characteristics as said peptide aptamer.

21. A pharmaceutical composition comprising a peptide aptamer, derivative thereof, or corresponding expressible nucleic acid, identified according to the method of claim 1, and a pharmaceutically acceptable carrier.

22. A method for treating a disease or condition associated with an abberant cell phenotype in a subject comprising:
administering to the subject, a therapeutically effective amount of a peptide aptamer, derivative thereof, or corresponding expressible nucleic acid, identified according to the method of claim 1.

23. The method of claim 22, wherein the abberant cell phenotype is associated with altered apoptosis, signal transduction, protein trafficking, cell adhesion, membrane transport, cell motility, or differentiation.

24. The method of claim 22, wherein the disease or condition is a cancer.

25. The method of claim 22, wherein the disease or condition is selected from the group consisting of osteoporosis and hematochromatosis.

26. The method of claim 22, wherein the expressible nucleic acid is administered using a retrovirus.

27. A peptide aptamer, derivative thereof, or corresponding expressible nucleic acid, identified according to the method of claim 1 in a form suitable for treating or inhibiting a disease or condition involving an abberant cell phenotype.

28. The peptide aptamer of claim 27, wherein the aberrant cell phenotype is associated with altered apoptosis, signal transduction, protein trafficking, cell adhesion, membrane transport, cell motility, or differentiation.

29. The peptide aptamer of claim 27, wherein the disease or condition is a cancer.

30. The method of claim 27, wherein the disease or condition is selected from the group consisting of osteoporosis and hematochromatosis.

31. A viral vector encoding a peptide aptamer suitable for treating a disease characterized by an abberant cell phenotype.

32. The viral vector of claim 31, wherein said misregulated cell phenotype is a cancer.

33. A kit for identifying a peptide aptamer capable of modifying a cell phenotype comprising:
a library of expressible nucleic acid sequences encoding peptide aptamers; and
instructions for use.

34. A kit for identifying a cancer phenotype comprising:
a library of expressible nucleic acid sequences encoding peptide aptamers; and instructions for use.
